(19) United States
(12) Patent Application Publication
(10) Pub. No.: US 2011/0166238 A1
(43) Pub. Date: Jul. 7, 2011

(54) Cobbs Company Aftershave Gel for Men & Women

(76) Inventors: Rufus Ree Cobbs, JR., Danville, VA (US); Joyce Elizabeth Valentine Cobbs, Danville, VA (US)

(21) Appl. No.: 12/941,968

(22) Filed: Nov. 8, 2010

Related U.S. Application Data
(60) Provisional application No. 61/335,216, filed on Jan. 4, 2010.

(51) Int. Cl. A61K 8/34; A61Q 19/00

(52) U.S. Cl. 514/724

(57) ABSTRACT

Cobbs Company Aftershave Gel is a cosmetic product designed to moisturize and maintain healthy looking skin. Cobbs Company Aftershave Gel can be applied to the skin area after shaving. Cobbs Company Aftershave Gel contains water, white petroleum jelly, and isopropyl alcohol. These three ingredients should only be mixed, sold, and used through the authorization of Cobbs Company.

### SUMMARY

Cobbs Company Aftershave Gel was created for the purpose of moisturizing and maintaining healthy looking skin after shaving. It should be understood that Cobbs Company Aftershave Gel is a cosmetic product.

### DESCRIPTION

Cobbs Company Aftershave Gel is a cosmetic product that is used to moisturize the skin to keep healthy skin looking smooth. Cobbs Company Aftershave Gel should be applied to the topical area that has been shaved. A small dime-sized amount is to be applied to the area after shaving. Cobbs Company Aftershave Gel can be applied daily or as needed.

### BACKGROUND

The process of making Cobbs Company Aftershave Gel is mixing water, white petroleum jelly, and isopropyl alcohol. Cobbs Company Aftershave Gel was created for the purpose of moisturizing and maintaining healthy looking skin after shaving. It should be understood that Cobbs Company Aftershave Gel is a cosmetic product.

1. A cosmetic product designed to moisturize and maintain healthy skin after shaving.

2. The cosmetic product contains water, white petroleum jelly, and isopropyl alcohol.

3. The three ingredients stated should be mixed, sold, and used only through the authorization of Cobbs Company.
